(19) United States
(12) Patent Application Publication
(10) Pub. No.: US 2014/0057074 A1
(43) Pub. Date: Feb. 27, 2014

(54) TEAR-RESISTANT AND ECO-FRIENDLY DISPOSABLE VISOR PROTECTIVE SHEETS

(71) Applicant: RICHARD ALEXANDER MCINTURFF, SANTA ANA, CA (US)

(72) Inventor: RICHARD ALEXANDER MCINTURFF, SANTA ANA, CA (US)

(21) Appl. No.: 14/071,243

(22) Filed: Nov. 4, 2013

Related U.S. Application Data
(63) Continuation of application No. 13/167,694, filed on Jun. 24, 2011.

(51) Int. Cl. A61F 9/02

(52) U.S. Cl. CPC A61F 9/025
USPC 428/136; 428/131

(57) ABSTRACT

A translucent tear-off lens is provided sufficiently resilient to permit a stack of such lenses to form fit over curvilinear protective eye wear, including goggles, where the translucent lens comprises a thin sheet of biodegradable material and tear-resistant means.

### RELATED APPLICATION

This continuation non-provisional application claims priority to non-provisional patent application U.S. Ser. No. 13/167,694 filed on Jun. 24, 2011, the entire contents of which is herein incorporated by reference.

### BACKGROUND

The embodiments herein relate generally to an improved protective sheet for use on eye shields that may be layered upon the shields for easy removal one at a time while resisting tearing at points where the sheets are retained to the shield and may be disposed in a manner that results in the sheet breaking down to environmentally tolerable and/or absorbable material.

For many years, motocross enthusiasts have enjoyed the thrill of riding off-road through circuits of meandering or looped roadways. Indeed, even a simple oval course seems to engender enthusiasm to speed for hours until one's clothes and body are covered in films of soil to varying degrees. Safety demands that a responsible rider wear some type of eye protection, normally eye goggles. The disadvantage, of course, is the amount of debris that can build up on the lens of the goggles over time. That problem is made even worse when the track is wet or even damp. The rotation of the bike wheels results in surprisingly large quantities of mud slinging rearwards, to the dismay of the trailing motorcyclists, whose goggles accumulate dirt in buckets full. The dirt build up diminishes the cyclists view, which not only impacts the cyclists ability to stay competitive, but it creates a hazardous situation both for him and the other cyclists.

As early as the 1970's, efforts have been made to overcome the problem of dirt build up on one's eye protective goggles, the most notable being what has become colloquially in the riding community as “tear-offs” or tear-off sheets. These are thin sheets of transparent thermoplastic material that can be stacked and layered upon the lens of one's goggles and removed one at a time as the top sheet becomes undesirably covered in soil and reduces vision. Indeed, U.S. Pat. No. 3,945,044 to McGee et al. dated Mar. 23, 1976 discusses the existence of such tear-offs and proposes, instead, a goggle frame with multiple auxiliary lenses that can be removed in real time while being worn. Issued in January of 1984, U.S. Pat. No. 4,428,081 to Smith discloses—as recited in the Abstract—an “Apparatus for providing a removable surface for protecting the lens of a . . . goggle [that] . . . includes a supply magazine for holding a protective film and a take-up magazine for receiving and holding spent film. The magazines are mounted oppositely and in spaced relation adjacent the lens to be protected. A film advance mechanism is provided for advancing the film from the supply magazine across the lens to a take-up magazine and a manual actuator is provided for the advancing mechanism.”

Numerous other prior art publications exist on the subject. U.S. Pat. No. 4,455,689 to Boyer, for example, discloses a bracket for controlling the release of tear-off sheets stacked on a set of goggles. U.S. Pat. No. 4,563,065 to Kreissl issued in January of 1986 discloses tear-off sheets with gripping tabs to facilitate removal of individual sheets stacked on top of the goggles lens. U.S. Pat. No. 4,716,601 to McNeal issued in January of 1988 discloses a system that facilitates the “rapid and easy mounting” of the sheets over a main goggle lens. U.S. Pat. No. 6,725,467 to Harding proposes in 2004 a device that combines both tear-off sheets and roll-on strips mounted on goggles.

Similar solutions have been proposed even for other types of protective eyewear, including welder's shields, as disclosed in U.S. Pat. No. 6,536,045 to Wilson et al. from March of 2003. Indeed, named inventor Hughes proposes in pending U.S. application Ser. No. 11/047,930, filed in February of 2005, a multi-layered lens protection system for safety glasses used in the painting industry, where painters frequently encounter paint splatter build-up on their glasses.

Although the above listed examples have enjoyed varying levels of success, tear-offs are ubiquitous in the sport of motocross where, depending upon the track conditions, a single rider may go through three to five tear-offs per race. At least two problems exist. One is presented by the question: where do all these tear-offs go? Usually, they land (and more problematically accumulate) on the ground or in the surrounding bushes and trees. Not surprisingly, the accumulation of tear-offs is aesthetically unpleasing. But more disconcerting is the potential safety hazard that may result from cyclists' wheels losing track grip while riding over discarded sheets of tear-off material.

Very little seems to have been done about this problem over the 30-odd years of use. Indeed, the problem has grown so dramatically over the years, some motocross racing hosts have either banned or are limiting the use of tear-off sheets by cyclists using their tracks or circuits. That's a significant problem for cyclists as few if any alternatives to tear-off sheets work effectively in practice.

The other problem alluded to above is that, even where permitted, tear-offs have some limitations in use. One of those limitations is the ability to resist a tendency to rip, leaving part of a soiled lens remaining on the protective goggles. Given how tightly individual tear-off sheets are stacked, it presents some difficulty for the rider in real time to remove the portion of the sheet that was not completely removed because it ripped. Traditionally, stacks of tear-off sheets are secured to protective eyewear in one of several possible ways, most commonly posts placed strategically at either side of the goggle lens. In many cases, a third post is provided either along the edge of the goggle lens support or on the leading edge of the strap that elastically wraps around the goggle wearer's head. The stacks of sheets, therefore, are provided with small holes positioned commensurate with the position of the posts on the goggles to permit secure retainment of the stack on the goggles during use. When tear-off sheets do rip upon removal, it is most often at the location of one of the posts.

Embodiments of the present invention solve at least these two problem with tear-offs, as described more fully below.

### SUMMARY

In embodiments of the present invention, materials are used that overcome the environmental and aesthetic issues associated with at least the prolific littering of tear-offs on and around motocross tracks, and other similar areas. Moreover, tear-resistant means are employed that minimize the circumstances of failed complete removal of individual tear-off sheets from a stack mounted onto the lens of protective eyewear. In that regard, one embodiment of the present invention comprises a translucent lens sufficiently resilient to permit a stack of such lenses to form fit over curvilinear protective eye wear, including goggles, in a manner that permits discrete removal of individual lenses when stacked on such protective eye wear, where the translucent lens comprises a thin sheet of material made of a biodegradable compound(s). One embodiment also includes first and second post mounts for securing one or more of such translucent lenses to the protective eye wear, wherein at least one of the post mounts comprises a plurality of holes connected therebetween by the tear-resistant means. In one embodiment, the tear-resistant means comprises a preformed slit extending between the plurality of holes of the at least one post mount.

The biodegradable material may be one of many types of materials, whether naturally occurring or synthetic, that decomposes on its own under natural ambient conditions. If desired, the biodegradable material may also be characterized by easy mechanical breakdown as well, for circumstances where the tear-off sheets fall onto the track of the circuit. When characterized by easy mechanical breakdown, cyclists riding over the tear-off sheet cause it to shred into smaller and more environmentally assimilating sizes. In that regard, the material may comprise one of several possible organic polymers or oligomers, including cellulose di-acetate.

### BRIEF DESCRIPTION OF THE FIGURES

The detailed description of some embodiments of the invention will be is made below with reference to the accompanying figures, wherein like numerals represent corresponding parts of the figures.

FIG. 1 shows one embodiment of the present invention comprising a single sheet of translucent biodegradable material having tear-resistant means;

FIG. 2 shows a stack of sheets of the embodiment of FIG. 1, where the stack is slightly askew to reveal the plurality of sheets within the stack;

FIG. 3 shows an example of protective eyewear that includes mounting posts on the lens of the protective eyewear to permit mounting of the stack of FIG. 2;

FIG. 4 shows an overhead view of a small stack of sheets of FIG. 1 mounted to the front face of the lens of protective eyewear of FIG. 3.

### DETAILED DESCRIPTION OF CERTAIN EMBODIMENTS

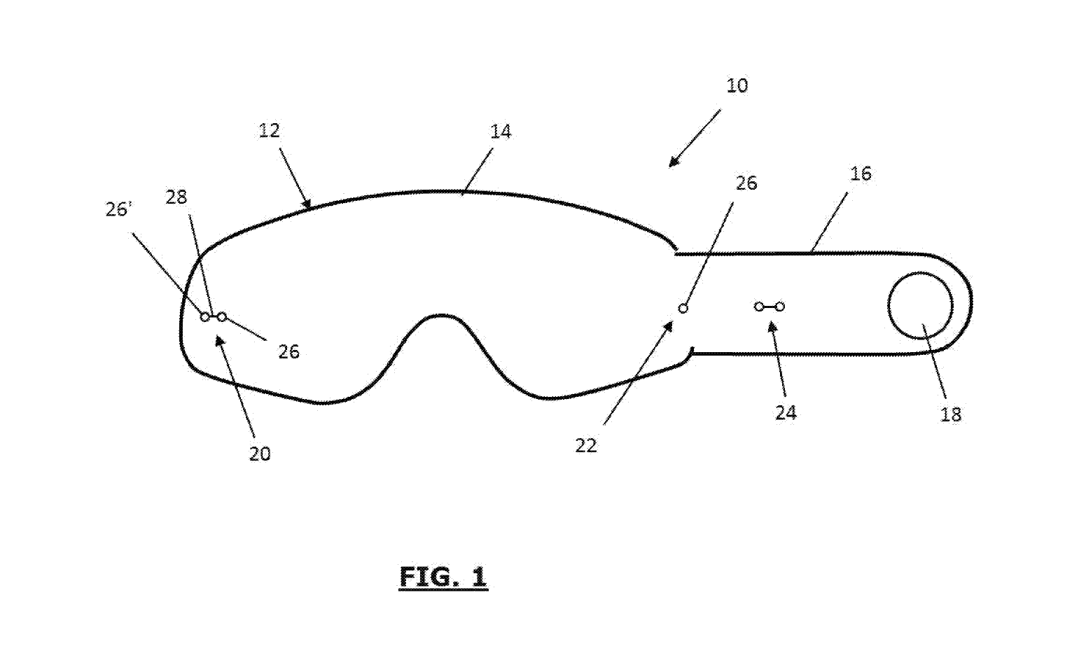

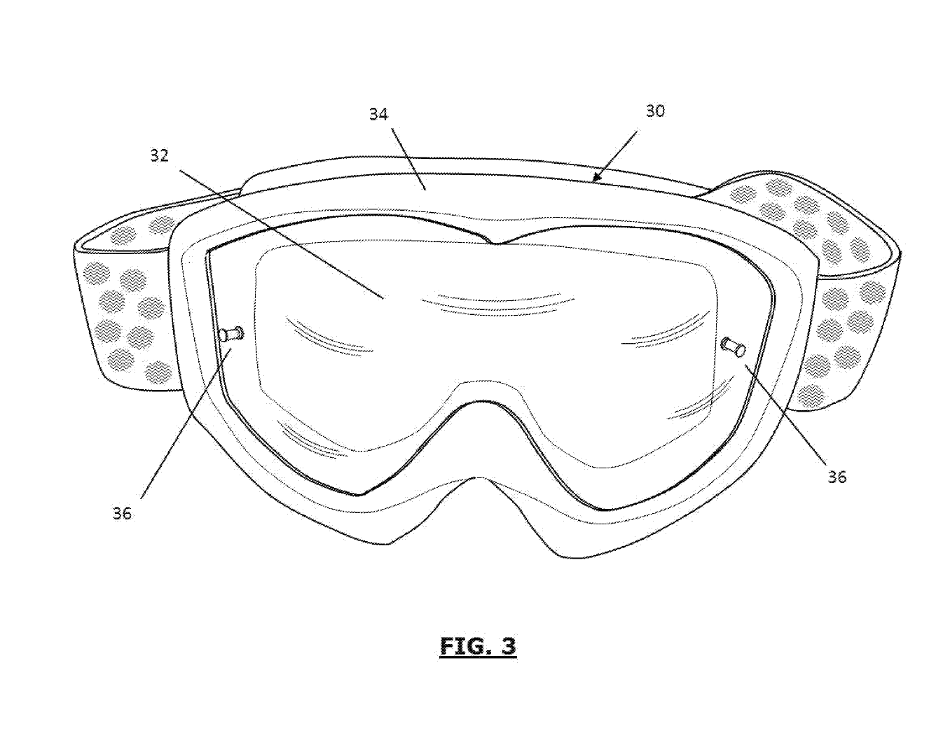

By way of example, and referring to FIG. 1, one embodiment of the present invention 10 comprises a translucent lens 12 made of a resilient thin sheet of thermoplastic material having a profile of one of numerous possibilities defined in part by the corresponding profile of the lens on protective eyewear to which the invention embodiments are directed. For example, FIG. 3 shows the type of protective eyewear known as a goggle that cyclists in motocross can use to shield their eyes from the debris kicked up by the tires of other cyclists as well as the dust that generally accumulates in the air during a race. The lens 12 of FIG. 1 bears a profile designed to mount to the goggle of FIG. 3. In that regard, the sheet 12 comprises a lens portion 14 and a pull tab portion 16 preferably manufactured integrally. The pull tab portion 16 presents a portion of material that extends outwardly away from the lens portion 14 to permit a user to grip it when desired to remove it from her protective eye wear. Optionally, a finger hole 18 is provided to facilitate the users securely gripping the lens 12 for quick removal while riding under obviously heavily distracted conditions.

As alluded to above, the translucent lens 12 is preferably made from naturally occurring or synthetic material that decomposes readily under natural ambient conditions, including, but not limited to, polyhydroxyalkanoates, such as the poly-3-hydroxybutyrate, polyhydroxyvalerates, polyhydroxyhexanoate, polylactic acid, polybutylene succinate, polycaprolactone, polyanhydrides polyvinyl alcohol, oligomers and polymers that are starch derivatives, and cellulose esters, such as cellulose acetate and nitrocellulose and their derivatives (celluloid). One desirable material specifically comprises cellulose di-acetate, which has been found to perform reliably in tests and satisfies the desired decomposing parameters.

Still referring to FIG. 1, the lens portion 14 comprises post mounts 20 and 22 on opposite ends of the lens portion 14, preferably to the periphery of the user's vision. Optionally, one embodiment may further comprise a third post mount 24 where the corresponding protective eye wear has more than two mounting posts. Each post mount 20, 22, 24 comprises at least one hole 26 in the sheet for preferably tight fit over corresponding mounting posts on the eye wear. Advantageously, the embodiment shown in FIG. 1 further comprises tear-resistant means positioned on at least one post mount, and preferably—although not necessarily—on the post mount farthest from the pull tab 16; namely, post mount 20. In that regard, tear-resistant means comprises a second hole 26′ positioned proximate hole 26 of post mount 20, with a pre-formed slit 28 extending between holes 26, 26′ of post mount 20. Typically, when tear-off is removed with a quick pulling action to reveal a clean tear-off below it, there is significant stress placed on the post mounts. It has been discovered through researching a variety of possible stress-relieving configurations, that a second hole placed proximate the post mounting hole coupled with a pre-formed slit will permit the tear-off sheet to be removed without tearing, or at least with minimal tearing that still results in the complete removal of the tear-off sheet form the protection eye wear.

In the embodiment of FIG. 1, for example, if the translucent lens 12 is mounted to eye wear such that hole 26 of post mount 20 is positioned over a corresponding mounting post on the eye wear, that when the lens 12 is pulled off from right to left (starting with the pull tab 16, that the eye wear post penetrating hole 26 of post mount 20 will, where the stress is heavy, simply slide through the pre-formed slit 28 and out secondary hole 26′, permitting complete removal of the sheet. If desired, third post mount 24 may also comprise tear-resistant means, including a secondary hole with pre-formed slit.

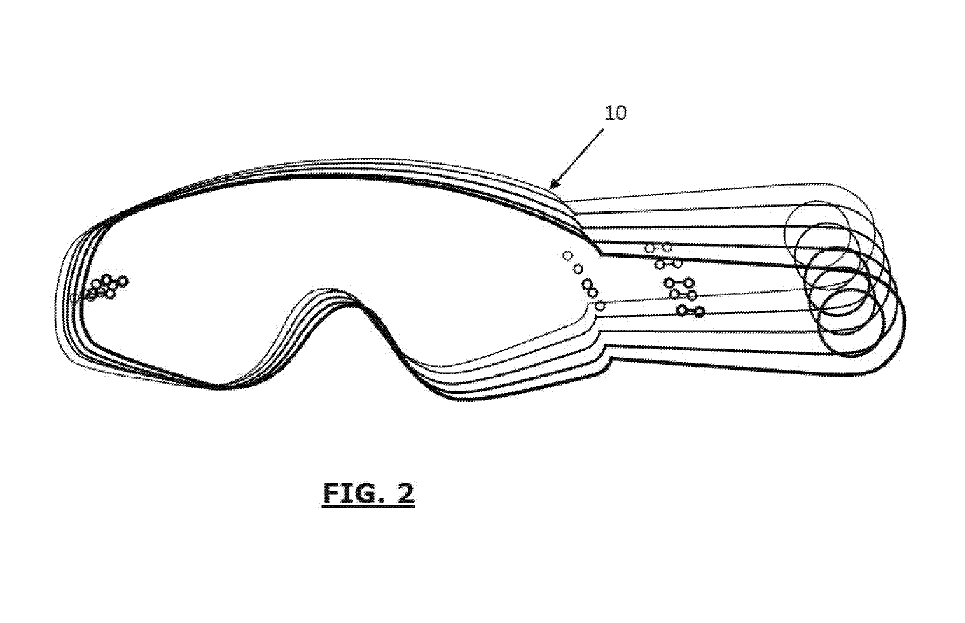

As explained above, the translucent lens 12 is preferably manufactured sufficiently thin and resilient to permit stacking of a plurality of such lenses 12 on top of each others, such as is shown in FIG. 2. Preferably, the plurality of lenses 12 are stacked such that the post mounts are aligned for secure mounting of the stack onto corresponding eye wear. In that regard, referring to FIG. 3, a typical embodiment of protective goggle 30 is presented comprising visor lens 32 supported by a cushioned frame 34. On opposite ends of the visor lens 32 are mounting posts 36. It is these posts, in this example, to which the holes 26 of post mounts 20 and 22 will engage with to secure a stack of translucent lens 12 to the goggle 30.

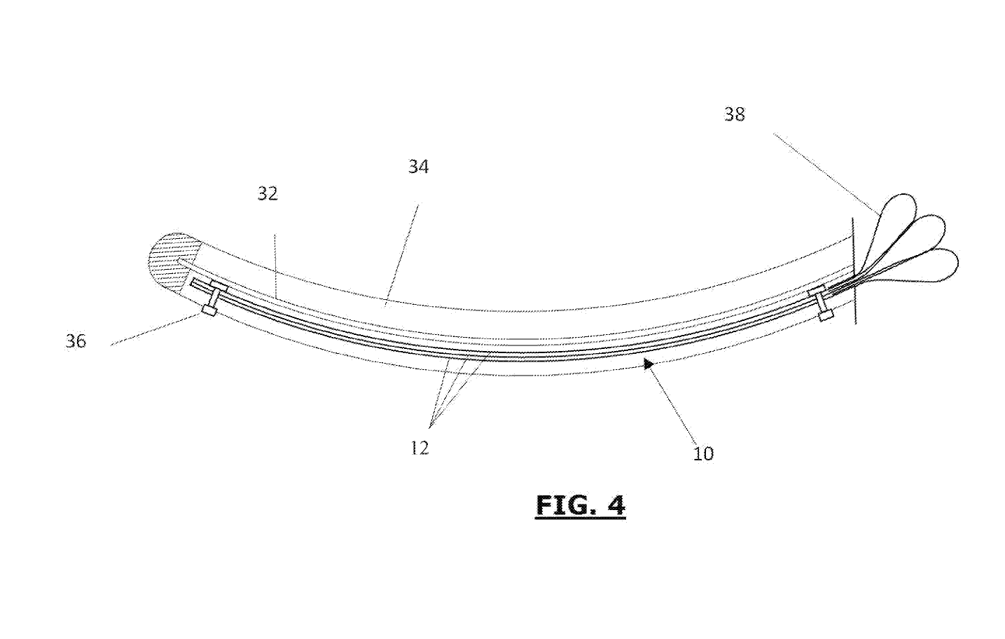

Referring to FIG. 4, embodiment 10 of FIG. 1 is shown stacked (in this case with three translucent lenses 12) in conforming fit to the visor lens 32 within frame 34 of goggle 30. The stack of translucent lens sheets are positioned so that the post mounts are secured to the mounting posts 36. Preferably, the individual lens sheets 12 are stacked whereby the pull tabs are folded against themselves (shown at lead line 38) and tucked between adjacent lens sheets to remain tightly therein until one at a time the user pulls one sheet off, exposing the underlying sheet.

The translucent lenses of the embodiments presented herein may be manufactured by one of many conventional techniques, including cutting a plurality of such lenses from a single sheet using a pattern that is overlaid (physically or electronically) over or across the sheet. Cutting may occur manually or automatically, such as with laser cutting means or the like. The sheet of material from which the lenses are cut is preferably sufficiently thin to provide a robust balance between strength and resilience.

Other embodiments of tear-off sheets are contemplated that incorporate the biodegradable advantage of the materials used as well as the tear-resistant features employed. For example, the post mount need not have any particular shape or size, and may actually comprises something other than a hole per se. Moreover, the tear-resistant means need not necessarily comprise both a secondary hole and pre-formed slit, but may comprise one or the other, or multiple secondary holes and/or slits as well. The profile of the translucent lens need not conform aesthetically to the generally shape of the corresponding visor lens on the eye wear, but may reflect more geometrical shapes as desired. Indeed, it is contemplated that fanciful shapes may be employed that function effectively to permit generally unimpeded vision to the rider while offering pleasing effects, including colors or tints, if so desired. It is further envisioned that logos may be emblazoned onto or cut into the lenses, including certification marks that signify to the user and industry that the translucent lenses are truly of acceptable biodegradable and/or tear-resistant quality. The scope of the invention, therefore, should not be limited by the embodiments illustrated and described expressly herein, but by the claims as allowed and set forth below.

What is claimed is:

1. A translucent lens sufficiently resilient to permit a stack of such lenses to form fit over curvilinear protective eye wear, including goggles, in a manner that permits discrete removal of individual lenses when stacked on such protective eye wear, the translucent lens comprising a thin sheet of biodegradable material, the lens further comprising first and second post mounts for securing one or more of such translucent lenses to the protective eye wear, wherein at least one of such post mounts comprises tear-resistant means.

2. The translucent lens of claim 1, wherein the biodegradable material comprises an organic polymer or oligomer from the group comprising polyhydroxyalkanoates, such as the poly-3-hydroxybutyrate, polyhydroxyvalerates, polyhydroxyhexanoate, polylactic acid, polybutylene succinate, polycaprolactone, polyanhydrides polyvinyl alcohol, oligomers and polymers that are starch derivatives, and cellulose esters, such as cellulose acetate and nitrocellulose and their derivatives.

3. The translucent lens of claim 2, wherein the biodegradable material comprises cellulose di-acetate.

4. The translucent lens of claim 1, wherein the post mounts comprise a hole through which a corresponding eye wear post may penetrate for securing the lens to the eye wear.

5. The translucent lens of claim 4, wherein the tear-resistant means comprises a pre-formed slit extending from the hole of the at least one post mount.

6. The translucent lens of claim 5, wherein the tear-resistant means comprises a secondary hole, whereby the pre-formed slit extends between the hole and the secondary hole.
